(19) United States
(12) Patent Application Publication
(10) Pub. No.: US 2024/0204133 A1
(43) Pub. Date: Jun. 20, 2024

(54) SEMICONDUCTOR LIGHT EMITTING ELEMENT

(71) Applicants: XIAMEN SILAN ADVANCED COMPOUND SEMICONDUCTOR CO., LTD., Xiamen (CN); HANGZHOU SILAN AZURE CO., LTD., Zhejiang (CN)

(72) Inventors: Jinjian Zheng, Xiamen (CN); Moran Gao, Xiamen (CN); Jingfeng Bi, Xiamen (CN); Weihong Fan, Xiamen (CN); Jiaming Zeng, Xiamen (CN); Chengjun Zhang, Xiamen (CN)

(21) Appl. No.: 18/554,856

(22) PCT Filed: Mar. 1, 2022
(86) PCT No.: PCT/CN2022/078635

§ 371 Date: Oct. 11, 2023

(30) Foreign Application Priority Data
Jun. 15, 2021 (CN) 202110662667.8

(51) Int. Cl. H01L 33/06; A61L 9/20; C02F 1/32; H01L 33/02; H01L 33/32

(52) U.S. Cl. CPC H01L 33/06; H01L 33/025; H01L 33/325; A61L 9/20; C02F 1/325; C02F 2303/04

(57) ABSTRACT

A semiconductor light emitting element is provided and includes: a substrate, an n-type semiconductor layer, a doped quantum well layer, a control layer for suppressing light attenuation with age and a p-type semiconductor layer from bottom to top. The control layer for suppressing light attenuation with age sequentially includes an undoped quantum well layer having at least one undoped barrier layer, a first control layer for suppressing light attenuation and/or a second control layer for suppressing light attenuation from bottom to top. The present disclosure reduces probability of Si of the doped quantum well layer and Mg of the p-type semiconductor layer diffusing and coming into contact with each other during long-term aging process, thereby suppressing light attenuation with age of the semiconductor light emitting element. A light attenuation of 1000 hours is reduced from 30% or higher (even 50% or higher) to within 10%.

### CROSS-REFERENCE TO RELATED APPLICATION

This application claims priority to the Chinese Patent Application No. 202110662667.8, filed on Jun. 15, 2021, entitled “semiconductor light emitting element”, which is incorporated herein by reference in its entirety in this disclosure.

### FIELD OF THE DISCLOSURE

The present disclosure relates to the field of semiconductor technologies, and in particular, to a semiconductor light emitting element.

### DESCRIPTION OF THE RELATED ART

An ultraviolet semiconductor light emitting element, having a wavelength range of 200-300 nm, emitting an ultraviolet light that is capable of breaking deoxyribonucleic acid (DNA) or ribonucleic acid (RNA) of viruses and bacteria, is capable of directly killing the viruses and the bacteria, and is widely used in a sterilization and disinfection fields of air purification, tap water sterilization, household air conditioner sterilization, automobile air conditioner sterilization and the like.

A well layer and a barrier layer of a quantum well layer of an existing ultraviolet semiconductor light emitting element have a lot of defects and poor quality, and may easily cause light attenuation with age; meanwhile, a distance between Si in the quantum well layer and Mg in a p-type semiconductor layer is too close, which easily causes migration and diffusion and mixing of Mg and Si under a condition of high temperature or long-term use, so that a light attenuation with age phenomenon is caused, and the light attenuation with age of 1000 hours is generally more than 30 percent, even more than 50 percent.

### SUMMARY OF THE DISCLOSURE

The present disclosure aims to provide a semiconductor light emitting element to improve performance of suppressing light attenuation with age of the semiconductor light emitting element, in particular an ultraviolet semiconductor light emitting element.

In order to achieve above and other related objects, the present disclosure provides a semiconductor light emitting element, comprising: a substrate, an n-type semiconductor layer, a doped quantum well layer, a control layer for suppressing light attenuation with age and a p-type semiconductor layer which are sequentially arranged from bottom to top, wherein the control layer for suppressing light attenuation with age comprises an undoped quantum well layer having at least one undoped barrier layer, a first control layer for suppressing light attenuation and/or a second control layer for suppressing light attenuation arranged from bottom to top sequentially.

Optionally, the control layer for suppressing light attenuation with age is not doped with Mg and Si.

Optionally, a sum of thicknesses of all barrier layers in the undoped quantum well layer is 4 nm to 12 nm.

Optionally, the undoped quantum well layer further comprises an undoped well layer, and the undoped well layer and the undoped barrier layer are alternately stacked to form the undoped quantum well layer.

Optionally, the doped quantum well layer is composed of undoped well layers and doped barrier layers, and the barrier layers of the doped quantum well layer have a Si doping concentration of 1E17 cm⁻³ to 5E19 cm⁻³.

Optionally, a material of the well layer of the doped quantum well layer comprises at least one of InₘGa₁₋ₘN and GaN, wherein m ranges from 0 to 0.3; a material of the barrier layer of the doped quantum well layer comprises AlₙGa₁₋ₙN, wherein n ranges from 0.1 to 1.

Optionally, a material of the first control layer for suppressing light attenuation comprises AlN.

Optionally, a thickness of the first control layer for suppressing light attenuation is 0.5 nm to 3 nm.

Optionally, a thickness of the second control layer for suppressing light attenuation is 0.5 nm to 5 nm.

Optionally, a material of the second control layer for suppressing light attenuation comprises Al_{z}Ga_{1-z}N, wherein z ranges from 0.2 to 1.

Optionally, the p-type semiconductor layer comprises a p-type electron barrier layer and a p-type contact layer on the p-type electron barrier layer, wherein a material of the p-type electron barrier layer comprises Al_{y}Ga_{1-y}N, wherein y ranges from 0.2 to 1; a material of the p-type contact layer comprises at least one of GaN and AlₖGa₁₋ₖN, wherein k is smaller than 0.45.

Optionally, a thickness of the p-type electron barrier layer is 15 nm to 50 nm; a Mg doping concentration of the p-type electron barrier layer is 1E19 cm⁻³ to 1E21 cm⁻³.

Optionally, a thickness of the p-type contact layer is 50 nm to 500 nm.

Optionally, an Al composition ratio of the second control layer for suppressing light attenuation is not less than an Al composition ratio of the p-type electron barrier layer.

Optionally, a material of the n-type semiconductor layer comprises AlₓGa₁₋ₓN, wherein x ranges from 0.1 to 1; a thickness of the n-type semiconductor layer is 1 μm˜3.5 μm; a Si doping concentration of the n-type semiconductor layer is 5E18 cm⁻³ to 5E19 cm⁻³.

Compared with a prior art, a technical scheme of the present disclosure has following beneficial effects:
according to the present disclosure, by adding the control layer for suppressing light attenuation with age between the doped quantum well layer and the p-type semiconductor layer, a probability of Si of the doped quantum well layer and Mg of the p-type semiconductor layer diffusing and coming into contact with each other during a long-term aging process is capable of being reduced, thereby improving the performance of suppressing light attenuation with age of the semiconductor light emitting element, in particular an ultraviolet semiconductor light emitting element, such that a light attenuation with age of 1000 hours is reduced from 30% or higher (even 50% or higher) to within 10%, and a long-term stability and the luminous efficiency of the semiconductor light emitting element are improved.

### BRIEF DESCRIPTION OF THE DRAWINGS

FIG. 1 is a schematic structural diagram of a semiconductor light emitting element according to an embodiment of the present disclosure;

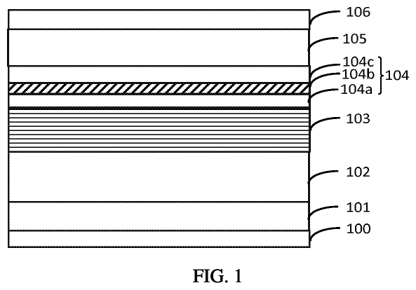

in FIG. 1:

100—substrate, 101—buffer layer, 102—n-type semiconductor layer, 103—doped quantum well layer, 104—control layer for suppressing light attenuation with age, 104a—undoped quantum well layer, 104b—first control layer for suppressing light attenuation, 104c—second control layer for suppressing light attenuation, 105—p-type electronic barrier layer, 106—p-type contact layer.

### DETAILED DESCRIPTION OF EMBODIMENTS OF THE DISCLOSURE

A well layer and a barrier layer of a quantum well layer of an existing ultraviolet semiconductor light emitting element have a lot of defects and poor quality, and may easily cause light attenuation with age; meanwhile, a distance between Si in the quantum well layer and Mg in a p-type semiconductor layer (e.g. an Al_{y}Ga_{1-y}N layer) is too close, which easily causes migration and diffusion and mixing of Mg and Si under a condition of high temperature or long-term use, so that a light attenuation with age phenomenon is caused, and the light attenuation with age of 1000 hours is generally more than 30 percent, even more than 50 percent.

In order to improve performance of suppressing light attenuation with age of a semiconductor light emitting element, in particular the ultraviolet semiconductor light emitting element, the disclosure provides a semiconductor light emitting element, wherein a control layer for suppressing light attenuation with age is added between a doped quantum well layer and the p-type semiconductor layer, a probability of Si of the doped quantum well layer and Mg of the p-type semiconductor layer diffusing and coming into contact with each other during a long-term aging process is capable of being reduced, thereby improving the performance of light attenuation with age of the semiconductor light emitting element, in particular the ultraviolet semiconductor light emitting element, such that the light attenuation with age of 1000 hours is reduced from 30% or higher (even 50% or higher) to within 10%, and a long-term stability and the luminous efficiency of the semiconductor light emitting element are improved.

The semiconductor light emitting element according to the present disclosure will be described in further detail with reference to drawings and specific examples. Advantages and features of the present disclosure will become more apparent from the following description. It is to be noted that the drawings are in a very simplified form and are not to precise scale, which is merely for the purpose of facilitating and distinctly claiming the embodiments of the present disclosure.

Referring to FIG. 1, the semiconductor light emitting element provided in this embodiment sequentially includes, from bottom to top: a substrate 100, an n-type semiconductor layer 102, a doped quantum well layer 103, a control layer for suppressing light attenuation with age 104 and a p-type semiconductor layer, wherein the control layer for suppressing light attenuation with age 104 sequentially includes an undoped quantum well layer 104a having at least one undoped barrier layer, a first control layer for suppressing light attenuation 104b and/or a second control layer for suppressing light attenuation 104c from bottom to top.

The substrate 100 may be one of a homogeneous or a heterogeneous substrate, including GaN, AlN, Ga₂O₃, SiC, Si, Sapphire, ZnO single crystal substrate, and refractory metal substrate with pre-deposited AlN film. Optionally, a substrate, such as a sapphire substrate, is used, which is capable of transmitting light emitted from the doped quantum well layer 103 and emitting an emitted light from a bottom side of the substrate. In order to improve light extraction efficiency, a surface of a light emitting side of the substrate 100 or a side opposing to the light emitting side may be in a concave-convex shape.

A buffer layer 101 may be formed on the substrate 100. The buffer layer 101 is used for reducing lattice mismatch between the substrate 100 and an epitaxial layer, so as to reduce a possibility of defects and dislocations occur in formed epitaxial layer, and improve crystal quality. The buffer layer 101 is not limited to one material, and may be a plurality of materials, combinations of different dopants and different doping contents, etc., and all materials of the buffer layer disclosed so far are within a scope of the present disclosure. Optionally, the material of the buffer layer 101 is nitride, for example, the material of the buffer layer 101 is AlN.

The n-type semiconductor layer 102 may be arranged on the substrate 100 based on the buffer layer 101 as needed, or the n-type semiconductor layer 102 may be arranged directly on the substrate 100. The n-type semiconductor layer 102 may be a conventional n-type layer, which is, for example, made of AlₓGa₁₋ₓN, wherein x ranges from 0.1 to 1. The n-type semiconductor layer 102 serves as an n-type layer by being doped with an n-type dopant, and as a specific example, the n-type dopant may be, but are not limited to, silicon (Si), germanium (Ge), tin (Sn), sulfur (S), oxygen (O), titanium (Ti), zirconium (Zr), and the like. A dopant concentration of the n-type dopant may be a dopant concentration at which the n-type semiconductor layer 102 is capable of serving as an n-type layer. Further, the n-type dopant in the n-type semiconductor layer 102 is optionally Si, and the doping concentration of Si is optionally 5E18 cm⁻³ to 5E19 cm⁻³. A thickness of the n-type semiconductor layer 102 is optionally 1 μm to 3.5 μm. In addition, a band gap of the n-type semiconductor layer 102 is optionally wider than that of a well layer of the doped quantum well layer 103 and has transmissivity to an emitted deep ultraviolet light. The n-type semiconductor layer 102 may have a single-layer structure or a multi-layer structure, or a superlattice structure.

The doped quantum well layer 103 is formed on the n-type semiconductor layer 102. The doped quantum well layer 103 may be a superlattice structure composed of InₘGa₁₋ₘN and AlₙGa_{1-N}, wherein m ranges from 0 to 0.3, n ranges from 0.1 to 1, and besides, the doped quantum well layer 103 may also be a superlattice structure composed of GaN and AlₙGa_{1-N}, but not limited thereto. For example, the doped quantum well layer 103 may be a superlattice structure composed of In_{0.2}Ga_{0.7}N and Al_{0.4}Ga_{0.6}N. The doped quantum well layer 103 is composed of an undoped well layer and a doped barrier layer, that is, the doped quantum well layer 103 generally includes a well layer and a barrier layer, for example, in a case of the doped quantum well layer 103 is the superlattice structure composed of InₘGa₁₋ₘN and AlₙGa_{1-N}, the well layer is InₘGa₁₋ₘN layer, the barrier layer is AlₙGa_{1-N} layer. A period number of the superlattice structure of the doped quantum well layer 103 is optionally 3 to 15, and the well layer and the barrier layer in each period form a pair of quantum wells. A thickness of each pair of quantum wells is optionally 6 nm to 20 nm. An In component ratio in the well layer of the doped quantum well layer 103 is optionally 0 to 0.3. The barrier layer of the doped quantum well layer 103, that is, Al_{0.4}Ga₁₋ₙN is doped with an N-type dopant (optionally Si), at an optional doping concentration of 1E17 cm⁻³ to 5E19 cm⁻³.

The control layer for suppressing light attenuation with age 104 is formed on the doped quantum well layer 103, and the control layer for suppressing light attenuation with age 104 sequentially includes the undoped quantum well layer 104a having at least one undoped barrier layer, the first control layer for suppressing light attenuation 104b and/or the second control layer for suppressing light attenuation 104c from bottom to top.

The undoped quantum well layer 104a and the doped quantum well layer 103 form a quantum well layer. Compared with the prior art, the barrier layers of last one or several of pairs of quantum wells in the quantum well layer in the embodiment are undoped barrier layers. In a case that the undoped quantum well layer 104a includes one undoped barrier layer, the barrier layer of last pair of quantum wells in the quantum well layer (i.e., the barrier layer of the pair of quantum wells closest to the first control layer for suppressing light attenuation 104b) is not doped with any n-type dopant. In a case that the undoped quantum well layer 104a includes at least two undoped barrier layers, the barrier layers of at least last two pairs of quantum wells in the quantum well layer (i.e., the barrier layers of at least two pairs of quantum wells closest to the first control layer for suppressing light attenuation 104b) are not doped with any n-type dopant, and in this case, the undoped quantum well layer 104a further includes an undoped well layer, i.e., the undoped well layer and the undoped barrier layer are alternately stacked to form the undoped quantum well layer 104a.

The barrier layer in the undoped quantum well layer 104a is optionally the same as the barrier layer of the doped quantum well layer 103 except for a difference in doping, for example, the barrier layer of the undoped quantum well layer 104a and the barrier layer of the doped quantum well layer 103 have a same thickness. The well layer of the undoped quantum well layer 104a is optionally the same as the doped quantum well layer 103. For example, a material of the well layer and barrier layer of the undoped quantum well layer 104a is optionally the same as that of the doped quantum well layer 103, that is, the undoped quantum well layer 104a may be the superlattice structure composed of InₘGa₁₋ₘN and AlₙGa_{1-N}, or the superlattice structure composed of GaN and AlₙGa_{1-N}. A period number of the superlattice structure of the undoped quantum well layer 104a is optionally 1 to 5.

A sum of thicknesses of all barrier layers in the undoped quantum well layer 104a is optionally 4 nm to 12 nm, and if the thickness of the all barrier layer in the undoped quantum well layer 104a is too thick, an injection of holes of the p-type semiconductor layer into the quantum well layer is affected, and if the thickness of the all barrier layer is too thin, contact between Si and Mg cannot be effectively blocked. The undoped quantum well layer 104a is capable of increasing a distance between Si in the doped quantum well layer 103 and Mg in the p-type semiconductor layer, and is helpful for reducing a probability of diffusion and contact between Si in the doped quantum well layer 103 and Mg in the p-type semiconductor layer in a long-term aging process, so as to improve the performance of light attenuation with age of the semiconductor light emitting element, especially an ultraviolet semiconductor light emitting element.

With continued reference to FIG. 1, the control layer for suppressing light attenuation with age 104 may further include the first control layer for suppressing light attenuation 104b disposed on the undoped quantum well layer 104a. A material of the first control layer for suppressing light attenuation 104b is optionally AlN, but is not limited thereto. A thickness of the first control layer for suppressing light attenuation 104b is optionally 0.5 nm to 3 nm. The first control layer for suppressing light attenuation 104b has a high potential barrier, and in order to effectively block electrons leakage to the p-type semiconductor layer and to block carriers from being trapped by defects, a thickness of which needs to be greater than 0.5 nm, but the thickness of which cannot exceed 3 nm, otherwise, holes are blocked from being injected into the doped quantum well layer 103, and a resistance and voltage are increased. The first control layer for suppressing light attenuation 104b is further capable of increasing the distance between Si in the doped quantum well layer 103 and Mg in the p-type semiconductor layer, which is helpful for reducing the probability of diffusion and contact between Si in the doped quantum well layer 103 and Mg in the p-type semiconductor layer in the long-term aging process, so as to improve the performance of light attenuation with age of the semiconductor light emitting element, especially the ultraviolet semiconductor light emitting element.

The control layer for suppressing light attenuation with age 104 further includes the second control layer for suppressing light attenuation 104c disposed on the first control layer for suppressing light attenuation 104b. A thickness of the second control layer for suppressing light attenuation 104c is optionally 0.5 nm to 5 nm. The second control layer for suppressing light attenuation 104c is made of Al_{z}Ga_{1-z}N, but not limited thereto, wherein z ranges from 0.2 to 1. An Al component ratio of the second control layer for suppressing light attenuation 104c is not lower than that of a p-type electron barrier layer 105, that is, z≥y, and the thickness cannot exceed 5 nm, otherwise, hole injection of the p-type electron barrier layer 105 into the doped quantum well layer 103 is blocked, and luminous efficiency is reduced. The second control layer for suppressing light attenuation 104c functions as an electron barrier layer to block electrons leakage to the p-type semiconductor layer, in addition to isolating Mg and Si from contacting each other. The second control layer for suppressing light attenuation 104c is further capable of increasing the distance between Si in the doped quantum well layer 103 and Mg in the p-type semiconductor layer, which is helpful for reducing the probability of diffusion and contact between Si in the doped quantum well layer 103 and Mg in the p-type semiconductor layer in the long-term aging process, so as to improve the performance of light attenuation with age of the semiconductor light emitting element, especially the ultraviolet semiconductor light emitting element.

The control layer for suppressing light attenuation with age 104 is not doped with Si and Mg, that is, the control layer for suppressing light attenuation with age 104 is an undoped structure. However, in actual tests, the control layer for suppressing light attenuation with age 104 had Si and Mg therein as measured by Secondary Ion Mass Spectrometry (SIMS), and Mg concentration was less than 5E19 cm⁻³, Si concentration was less than 1E17 cm⁻³, mainly because Mg in a Mg-doped p-type semiconductor layer and Si in a Si-doped quantum well layer diffuse to the control layer for suppressing light attenuation with age 104.

The control layer for suppressing light attenuation with age 104 is capable of reducing the probability of diffusion and contact between Si in the doped quantum well layer 103 and Mg in the p-type semiconductor layer in the long-term aging process, thereby improving the performance of light attenuation with age of the ultraviolet semiconductor and reducing the light attenuation with age of 1000 hours from more than 50% to less than 10%.

The control layer for suppressing light attenuation with age 104 may also be applied to semiconductor light emitting elements of all other wavelength ranges with the wavelength range of 200 nm to 550 nm, such as deep ultraviolet semiconductor light emitting elements, ultraviolet semiconductor light emitting elements, violet semiconductor light emitting elements, blue semiconductor light emitting elements, green semiconductor light emitting elements, and yellow semiconductor light emitting elements.

The p-type semiconductor layer is disposed on the control layer for suppressing light attenuation with age 104, and the p-type semiconductor layer may include the p-type electron barrier layer 105 and a p-type contact layer 106. The p-type electron barrier layer 105 is used for blocking electrons, preventing the electrons from overflowing to the p-type contact layer 106, and further injecting the electrons into the doped quantum well layer 103, so that occurrence of non-radiative recombination is reduced, and the luminous efficiency of the semiconductor light emitting element, especially the ultraviolet semiconductor light emitting element, is further improved.

A material of the p-type electron barrier layer 105 is optionally Al_{y}Ga_{1-y}N, but not limited thereto, wherein y ranges from 0.2 to 1. A thickness of the p-type electron barrier layer 105 is optionally 15 nm to 50 nm. Examples of p-type dopants doped into the p-type electron barrier layer 105 include, but are not limited to, magnesium (Mg), zinc (Zn), calcium (Ca), beryllium (Be), and manganese (Mn). The p-type dopant is optionally Mg. A dopant concentration of the p-type electron barrier layer 105, e.g., a Mg doping concentration, is optionally 1E19 cm⁻³ to 1E21 cm⁻³.

The p-type contact layer 106 is disposed on the p-type electron barrier layer 105. The p-type contact layer 106 is a layer used for reducing contact resistance between a p-side electrode arranged right above which and the p-type electron barrier layer 105. A material of the p-type contact layer includes at least one of GaN and AlₖGa₁₋ₖN, but not limited thereto, wherein k<0.45. As the p-type contact layer of the semiconductor light emitting element, a GaN layer which easily increases a hole concentration is generally used, and an AlₖGa₁₋ₖN layer (an Al component ratio k<45%) may also be used, although the AlₖGa₁₋ₖN layer may have a slightly reduced hole concentration compared to the GaN layer, since light emitted from the light emitting layer, for example, ultraviolet light, is capable of penetrating the AlₖGa₁₋ₖN layer, an overall light extraction efficiency of the semiconductor light emitting element, particularly the ultraviolet semiconductor light emitting element, is improved, and a light output of the semiconductor light emitting element, particularly the ultraviolet semiconductor light emitting element is improved. A thickness of the p-type contact layer 106 is 50 nm to 500 nm.

In the semiconductor light emitting element of the embodiment, the undoped control layer for suppressing light attenuation with age is added between the doped quantum well layer and the p-type semiconductor layer, so that the probability of diffusion and contact between Si in the quantum well layer and Mg in the p-type semiconductor layer in the long-term aging process is reduced, thereby improving the performance of light attenuation with age of the semiconductor light emitting element, especially the ultraviolet semiconductor light emitting element, such that the light attenuation with age of 1000 hours is reduced from 30% or higher (even 50% or higher) to within 10%, and a long-term stability and the luminous efficiency of the semiconductor light emitting element are improved.

The above description is only for the purpose of describing the optional embodiments of the present disclosure, and is not intended to limit a scope of the present disclosure, and any variations and modifications made by those skilled in the art based on the above disclosure are within the scope of appended claims.

What is claimed is:

1. A semiconductor light emitting element, comprising: a substrate, an n-type semiconductor layer, a doped quantum well layer, a control layer for suppressing light attenuation with age and a p-type semiconductor layer which are sequentially arranged from bottom to top, wherein the control layer for suppressing light attenuation with age comprises an undoped quantum well layer having at least one undoped barrier layer, a first control layer for suppressing light attenuation and/or a second control layer for suppressing light attenuation arranged from bottom to top sequentially.

2. The semiconductor light emitting element according to claim 1, wherein the control layer for suppressing light attenuation with age is not doped with Mg and Si.

3. The semiconductor light emitting element according to claim 1, wherein a sum of thicknesses of all barrier layers in the undoped quantum well layer is 4 nm to 12 nm.

4. The semiconductor light emitting element according to claim 1, wherein the undoped quantum well layer further comprises an undoped well layer, and the undoped well layer and the undoped barrier layer are alternately stacked to form the undoped quantum well layer.

5. The semiconductor light emitting element according to claim 1, wherein the doped quantum well layer is composed of a plurality of pairs of undoped well layers and doped barrier layers, and the barrier layers of the doped quantum well layer have a Si doping concentration of 1E17 cm⁻³ to 5E19 cm⁻³.

6. The semiconductor light emitting element according to claim 5, wherein a material of the well layer of the doped quantum well layer comprises at least one of InₘGa₁₋ₘN and GaN, wherein m ranges from 0 to 0.3; a material of the barrier layer of the doped quantum well layer comprises AlₙGa_{1-N}, wherein n ranges from 0.1 to 1.

7. The semiconductor light emitting element according to claim 1, wherein a material of the first control layer for suppressing light attenuation comprises AlN.

8. The semiconductor light emitting element according to claim 1, wherein a thickness of the first control layer for suppressing light attenuation is 0.5 nm to 3 nm.

9. The semiconductor light emitting element according to claim 1, wherein a thickness of the second control layer for suppressing light attenuation is 0.5 nm to 5 nm.

10. The semiconductor light emitting element according to claim 1, wherein a material of the second control layer for suppressing light attenuation comprises Al_{z}Ga_{1-z}N, wherein z ranges from 0.2 to 1.

11. The semiconductor light emitting element according to claim 10, wherein the p-type semiconductor layer comprises a p-type electron barrier layer and a p-type contact layer on the p-type electron barrier layer, wherein a material of the p-type electron barrier layer comprises Al_{y}Ga_{1-y}N, wherein y ranges from 0.2 to 1; a material of the p-type contact layer comprises at least one of GaN and AlₖGa₁₋ₖN, wherein k is smaller than 0.45.

12. The semiconductor light emitting element according to claim 11, wherein an Al composition ratio of the second control layer for suppressing light attenuation is not less than an Al composition ratio of the p-type electron barrier layer.

13. The semiconductor light emitting element according to claim 11, wherein a thickness of the p-type electron barrier layer is 15 nm to 50 nm; a Mg doping concentration of the p-type electron barrier layer is 1E19 cm⁻³ to 1E21 cm⁻³.

14. The semiconductor light emitting element according to claim 11, wherein a thickness of the p-type contact layer is 50 nm to 500 nm.

15. The semiconductor light emitting element according to claim 1, wherein a material of the n-type semiconductor layer comprises AlₓGa₁₋ₓN, wherein x ranges from 0.1 to 1; a thickness of the n-type semiconductor layer is 1 μm˜3.5 μm; a Si doping concentration of the n-type semiconductor layer is 5E18 cm⁻³ to 5E19 cm⁻³.
